(19) United States
(12) Patent Application Publication
(10) Pub. No.: US 2014/0279600 A1
(43) Pub. Date: Sep. 18, 2014

(54) AUTOMATED MONITORING OF PEST TRAPS IN A DISTRIBUTED WORK ENVIRONMENT

(71) Applicant: Mitchell Barry Chait, Las Vegas, NV (US)

(72) Inventor: Mitchell Barry Chait, Las Vegas, NV (US)

(21) Appl. No.: 13/837,852

(22) Filed: Mar. 15, 2013

(51) Int. Cl. G01N 33/00; G06Q 50/04; G06Q 30/00; G06T 7/00

(52) U.S. Cl. CPC G01N 33/00; G06T 7/0004; G06Q 50/04; G06Q 30/018
USPC 705/317; 250/338.1; 250/349; 702/19; 382/110

(57) ABSTRACT

Techniques for automatically monitoring and reporting pest infestation in a distributed work environment, and resolving detected non-compliances. Sensors are configured to collect various types of data in the distributed work environment, including data related to pest traps and data related to human inspection and handling of pest traps. A rules engine analyzes the data collected from the sensors to determine compliance with a set of rules related to pest infestation. Other types of data that may be collected by sensors and analyzed by the rules engine include machine data, biological data, and environmental data. The rules may include governmental regulations, industry standards, and company specifications. If the rules engine detects existing non-compliance and/or potential non-compliance in the collected data with at least one of the rules, it generates alerts, remediation instructions, and/or takes actions to resolve the non-compliance.

### BACKGROUND

There are numerous safety concerns that surround distributed work environments. For example, in the context of the commercial food industry, food production facilities must ensure the safety of consumers who consume the food products, as well as the safety of workers who work in the facilities. Various rules and standards have been developed by various entities to regulate the safety of operations within, as well as goods/services produced by, distributed work environments. For example, safety measures in the food industry typically regulate practices such as refrigeration, biosecurity, cleaning, disinfection, and pest control. Pests, such as rodents, pose numerous health hazards to the food industry. As an example, egg farms are typically required to comply with various rules and regulations to ensure the safety of shell eggs produced for consumption by the public.

Among the numerous health hazards posed by pests in the food industry, rodents are known to be potential carriers of many diseases, including salmonellosis, pasteurellosis, leptospirosis, trichinosis, toxoplasmosis and rabies. In particular, Salmonella is responsible for numerous human illnesses and deaths per year. Many Salmonella illnesses are attributed to contaminated eggs produced in egg-laying and egg-handling environments. Salmonella can contaminate eggs by direct contact between contaminated material and the eggs, or by infecting the hens that lay the eggs. Egg-laying hens can ingest Salmonella through contaminated feed, for example, and consequently lay eggs that are contaminated with Salmonella. Eggs that are contaminated with Salmonella may then be passed on to human consumers, causing illness and potentially death.

### SUMMARY

Ensuring the safety and cleanliness of a distributed work environment can be a challenge. For example, in the egg industry, a typical egg farm may have up to tens of thousands of hen cages, with various feeding stations and egg nests, that must be monitored and maintained for cleanliness. A significant part of ensuring cleanliness involves monitoring rodent infestation. An egg farm typically relies on human workers to inspect up to hundreds of rodent traps that are distributed throughout the egg farm. However, human inspection can be labor-intensive and poses risks to workers traversing unsanitary and cramped conditions in an egg farm. Furthermore, such undesirable conditions can cause human inspection to be prone to errors and/or fraud.

Egg farms are typically limited in their accuracy and promptness of detecting and handling pest infestation by the actions of human workers. Without accurate monitoring and prompt handling of rodent infestation, the egg industry can pose a danger to egg-laying hens, workers, and human consumers.

The inventors have recognized and appreciated that improvements in the quality and safety of a distributed work environment may be achieved by a system that automatically monitors, in a real-time manner, a plurality of pest traps, issues alerts and/or instructions based on detected activation of traps, and takes action to resolve non-compliance with respect to pest infestation. In some embodiments, one or more sensors may be distributed throughout a work environment and be configured to collect data related to the locations and/or number of pests trapped.

The inventors have also recognized and appreciated that various types of sensors may be used in combination to monitor pest traps, and that the sensors may be coordinated with other systems in the distributed work environment to facilitate detection of traps. For example, in egg farms that utilize an illumination system designed to stimulate egg production from hens, one or more trap sensors may be coordinated with the timing of the illumination system.

The system may analyze data from one or more types of sensors in various ways to determine and/or predict a level of pest infestation, without necessarily relying on human labor to physically inspect traps and report data. For example, in some embodiments, the system may cross-correlate different types of data. The data that is collected and analyzed may relate to the rodent traps themselves and, in some embodiments, may also relate to other aspects of the egg farm, such as machines, operations, ambient environment, human workers, or any other suitable aspect of the egg farm. Such a system may enable intelligent, automated, and holistic monitoring of an egg farm to accurately detect and/or predict a level of pest infestation.

The results of the system analysis may be used by any suitable entity, such as the egg farm itself, government regulators, customers of the egg farm, etc. In some embodiments, the trap monitoring system may be used as part of a larger compliance-monitoring system, involving multiple different entities.

As a non-limiting example, commercial egg farms are typically required to monitor and control pest infestation in and around egg-laying hens. Such requirements may be mandated by governmental rules, customer specifications, and/or in-house standards within the egg farm itself. For example, a typical rodent monitoring program includes monitoring rodents through visual inspection and mechanical traps, and when undesirable levels of rodent infestation are found, to take corrective action to achieve compliance with one or more rules.

One embodiment is directed to at least one sensing device configured to monitor a pest trap, the at least one sensing device comprising a microsensor configured to detect pest infestation information; and a transmitter configured to transmit the pest infestation information.

Another embodiment is directed to A system of monitoring pest traps, the system comprising: at least one sensing device configured to monitor a pest trap; and a computing device configured to determine, based on sensor information received from the at least one sensing device, a compliance status.

Another embodiment is directed to at least one computer-readable medium having stored thereon computer-readable program instructions which, when executed by at least one processor, perform acts of receiving sensor data indicating activation of a pest trap; processing at least part of the sensor data; determining, based on the processing of at least part of the sensor data, whether the sensor data satisfies at least one rule; and outputting at least one result based on determining whether the sensor data satisfies the at least one rule.

Another embodiment is directed to a system configured to monitor, manage, and instrument compliance in a distributed work environment. The system comprises at least one input configured to receive data comprising pest infestation information and secondary information. The secondary information is related to one or more of: behavior of one or more persons responsible for taking action in the distributed work environment; biological or environmental parameters associated with the distributed work environment; operational conditions and/or events; apparatus usage and/or condition; at least one standard and degree of compliance therewith; or product production and/or delivery logistics. The system also comprises a data store configured to store the data, and at least one processor configured to execute stored program instructions to process at least part of the data and determine, based on the processing of at least part of the data, a compliance status

Another embodiment is directed to a system configured to monitor, manage, and instrument compliance in a distributed work environment. The system comprises: at least one input configured to receive data comprising pest infestation information and secondary information related to one or more of: behavior of one or more persons responsible for taking action in the distributed work environment; biological or environmental parameters associated with the distributed work environment; operational conditions and/or events; apparatus usage and/or condition; at least one standard and degree of compliance therewith; or product production and/or delivery logistics. The system further comprises a data store configured to store the data, and at least one processor configured to execute stored program instructions to process at least part of the data and determine, based on the processing of at least part of the data, a compliance status.

It should be appreciated that all combinations of the foregoing concepts and additional concepts discussed in greater detail below (provided that such concepts are not mutually inconsistent) are contemplated as being part of the inventive subject matter disclosed herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic illustration of an example of a system of automated monitoring and reporting of pest infestation in which some embodiments may be implemented;

FIG. 2 is a schematic illustration of an example of an environment in which a system of automated monitoring and reporting of pest infestation may be used, in accordance with some embodiments;

FIG. 3 is a schematic illustration of an example of a data store configured to store information including sensor data and rules, in accordance with some embodiments;

FIG. 4 is a flow chart of an example of processing performed by a system of automated monitoring and reporting of pest infestation, in accordance with some embodiments;

FIG. 5 is a flow chart of an example of processing performed by a rules engine in a system of automated monitoring and reporting of pest infestation, in accordance with some embodiments; and

FIG. 6 is an example of a computing system on which some embodiments may be implemented.

### DETAILED DESCRIPTION

The inventors have recognized and appreciated that significant advances in the quality and safety of food products produced in food production facilities may be achieved by a system that automatically monitors and analyzes data related to pest traps in the food production facilities, and takes action to resolve detected non-compliance. In some embodiments, results of the analysis may be reported to one or more reporting devices, indicating locations and/or numbers of traps that may have pests. The inventors have recognized and appreciated that an automated pest trap monitoring system may facilitate accurate and fast detection of pests in a food production facility, and thus reduce the likelihood of contamination and improve the safety of consumers of the food and workers in the facility.

The inventors have recognized and appreciated that an automated trap monitoring and alert system in a food production facility may improve safety of human workers in the food production facility by reducing the need for human labor to inspect the traps in potentially dangerous and unsanitary conditions. As a non-limiting example, a commercial egg farm may comprise one or more hen houses potentially distributed over a wide geographic area, with up to thousands of traps in and around various facilities. It is often desirable or required to inspect traps on a frequent basis, in some cases as often as daily. However, it may be difficult to ensure that workers accurately and consistently inspect all the traps in an egg farm. Furthermore, it may also be difficult for regulators or customers to verify that a pest count, such as a rodent index, reported by the egg farm is indeed accurate.

It should be appreciated, however, the embodiments are not limited to trapping and counting rodents in an egg farm, as any suitable type of pest may be trapped and automatically monitored and reported in any suitable food production facility, not necessarily limited to those producing eggs. For example, the food production facility may be any facility related to food production and distribution, including but not limited to a warehouse, a consumer market, or a restaurant.

An automated trap monitoring system may report results of the analysis to any suitable entity, such as the food production facility and/or third-parties, such as governmental inspectors or customers of the food production facility. The inventors have recognized and appreciated that, by automating the detection, localization, and reporting of trapped infestation, food production facilities and/or third-parties may reduce their reliance on and/or supplement potentially inaccurate and/or fraudulent human activities to inspect, count, and report pest infestation.

In some embodiments, the system may enable a food production facility to proactively detect and eliminate potentially contaminated food products, based on detected level and/or location of pest infestation. As such, the food production facility may be able to reduce the probability of distributing contaminated food products to the general consumer public, and thus reduce the various undesirable consequences of distributing contaminated food products, such as governmental penalties, food product recalls, and loss of customer confidence. The inventors have recognized and appreciated that such a system may improve efficiency, safety (for workers and consumers), and reduce costs in the food production industry.

In some embodiments, an automated trap monitoring and reporting system may be used as part of a larger system that monitors and analyzes various types of data, of which trap information may just be one type of data, to determine compliance of a food production facility. In some embodiments, the trap monitoring system, or a larger system comprising the trap monitoring system, may take one or more actions to resolve detected non-compliance, such as generating alerts, instructions, or re-configuring operations of the food production facility. Though, it should be appreciated that regardless of whether the trap monitoring system is used as part of a larger system, the trap monitoring system may use various types of information related to pest traps to determine non-compliance in a food production facility, and take action(s) to resolve non-compliance.

The pest traps may be any suitable type of trap, placed at any appropriate locations in a food production facility. It should be appreciated that embodiments are not limited to any particular type, number, or locations of traps. In some embodiments, a pest trap may be a rodent trap configured to trap one or more rodents. For example, a trap may comprise a looking glass which may allow external inspection, for example, either directly by a human worker or by a sensor device, such as a camera. Alternatively or additionally, a trap may comprise one or more indication techniques that provides indication(s) of trapped pests. As an example, a low-voltage light emitting diode (LED) may indicate when the trap is activated. It should be appreciated, however, that embodiments are not limited any particular type of trap indicator, as indicators may be visual, audible, tactile, or use any suitable technique to indicate presence or amount of trapped infestation.

One or more sensors may be configured to monitor the traps. Sensors may be attached to the traps or remote from the traps. The sensors may collect any suitable type(s) of data related to pest infestation. As non-limiting examples, sensors may detect information related to number(s) and/or location(s) of trapped pests. The information may be collected by any suitable technique, examples of which include, but are not limited to, mechanical activation of traps, a weight of traps, movement in traps, or any other suitable information related to pests that may be captured in traps. Sensors may comprise any suitable number of components, such as a camera, one or more actuators, remote motion detectors, or other suitable components that enable collection of data related to determining a number of trapped pests.

In some embodiments, in addition to trap sensors, other types of sensors may be used to collect a variety of data throughout the food production facility that may be related to pest infestation. For example, if the food production facility includes an egg farm, then some sensors may be configured to monitor locations where pests may be particularly active, such as areas containing food and water, or sensors may be configured to monitor activity of hens to detect disturbances that may be caused by pests. It should be appreciated, however, that embodiments are not limited to any particular any a particular type of food production facility, nor any particular type, number, or location(s) of sensors in the food production facility.

The inventors have recognized and appreciated that, in some embodiments, if different types of data are collected and analyzed from different sensors, then the reliability and accuracy of pest detection may be improved. For example, if data related to hens and/or the ambient environment of the food production facility are collected, in addition to data related to traps, then this may add redundancy and reliability to pest detection and location determination. In some embodiments, this may not only improve positive detection of pests but may also reduce occurrences of “false alarms” and therefore reduce the need to send human workers to inspect empty traps in potentially unsanitary and unsafe conditions in and around the food production facility.

In some embodiments, sensors may be coordinated with one or more systems particular to the environment. In the example of an egg farm, sensors may be coordinated with systems configured to control and/or facilitate egg-laying by hens. For example, some egg farms utilize an automated illumination system configured to illuminate only certain parts of an egg farm at certain times, to regulate the times at which different hens lay eggs. In such scenarios, sensors may be configured to collect data in coordination with the illumination system. It should be appreciated, however, that embodiments are not limited to coordinating sensors with illumination systems, as sensors may be coordinated with any suitable system in any suitable food production facility.

In some embodiments, the sensors may transmit the collected data to one or more computing devices for storage and/or analysis. For example, the computing devices may be central servers, though embodiments are not limited in this regard, as any suitable computing device (such as personal computers or portable computing devices) may be used to store and process the data. The server(s) may execute one or more algorithms that implement rules to determine a number and/or location of trapped pests, based on the data collected by the sensors and one or more rules or specifications. The analysis may be performed on any suitable type of collected data related to pest infestation, including but not limited to number of activations in a trap, the total weight inside a trap, an image processing of an area inside or around the trap, etc.

In some embodiments, the location(s) of potentially trapped pests may be determined based on the collected data. For example, the location(s) may be determined by receiver triangulation and/or a database of trap/sensor locations. Additionally or alternatively, location data may be transmitted by the sensors. It should be appreciated, however, that embodiments are not limited to any particular technique of location determination, as any suitable technique(s) may be used to determine the location(s) of potentially trapped pests based on sensor data from the food production facility.

In some embodiments, one or more reporting devices may receive and indicate instructions and/or alerts regarding potentially trapped pests, based on the analysis of the data collected by sensors. The reporting devices may be used, for example, by a supervisor, workers, or other suitable entity, who may be notified of a number and/or location of trapped pests. In some embodiments, this reporting may occur nearly instantaneously, to reduce the delay of removing potentially disease-carrying infestation.

The reporting devices may be communicative with the server(s) via a communication link or network, and may be local to or remote from the food production facility. It should be appreciated, however, that separate reporting devices are optional, as indications and/or alerts may be generated on any suitable computing device(s). As non-limiting examples, indications and/or alerts may be generated on the computing device(s) processing the collected data, on the one or more sensors themselves (e.g., using visible alerts, audible alarms, etc.), or on any other suitable device, whether local or remote to the food production facility.

The inventors have recognized and appreciated that such an automated trap monitoring and reporting system may enable efficient, accurate, and proactive detection of pest infestation in a food production facility. Particularly in large food production facilities with potentially hundreds of thousands of components and entities, such a system may mitigate difficulties in ensuring the safety and compliance of food production facilities with respect to various regulations and specifications related to pest infestation. A typical food production facility may be subject to various different types of requirements, based on governmental, industrial, or customer standards. In some embodiments, the system may enable automated integration and management of multiple different rules and specifications, and implement any changes in real-time.

The inventors have recognized and appreciated that an automated pest trap monitoring and reporting system may be useful in a wide variety of food production facilities. For example, if the food production facility comprises an egg farm, then the system may be utilized in egg farms including those using battery cages or those in which hens are allowed to roam freely around an indoor or outdoor enclosure. Regardless of the exact nature of the egg farm, a system that automatically monitors pest infestation, using one or more sensors, and determines the location(s) and/or number of trapped pests using suitable estimation algorithms, may improve the efficiency and safety of egg production.

An automated monitoring and reporting system may reduce the need for workers to manually check potentially unsafe and unsanitary conditions for trapped pests. In some embodiments, the system may also be used in conjunction with human monitoring, and may reduce inaccuracies and/or fraud in the monitoring of infestation.

In some embodiments, the inventors have recognized and appreciated that the system may enable proactive management of workers. For example, different workers may be monitored while performing particular tasks to determine whether they are performing the tasks pursuant to a set of instructions and/or standards. The system may be able to automatically detect error and/or fraud by correlating human behavioral data with other types of sensor data. As a non-limiting example, if a worker tries to manipulate machine records in a manner that is inconsistent with data collected by human behavioral sensors and/or machine sensors, then the system may detect the non-compliance and alert an appropriate person or entity.

The inventors have recognized and appreciated that such a system may mitigate difficulties in controlling a multitude of workers and/or entities in a distributed work environment. In some embodiments, the system may be provided with, or may dynamically learn, the tasks assigned to different workers and, based on collected data, may dynamically learn the capabilities of the workers. The inventors have recognized and appreciated that such a system may improve efficiency and productivity by enabling improved coordination and allocation of resources among different workers and/or entities, providing faster and more accurate decision-making and responses to problems.

In some embodiments, human behavioral data may be correlated with other types of data to detect patterns of inconsistency, error, and/or fraud that may indicate non-compliance with instructions or standards. In some embodiments, the standards may comprise rules and/or instructions provided by a suitable entity, such as a governmental agency, an industry group, and/or a specific company. The system analyze and correlate data collected from potentially diverse sensors. Such an integrated system may enable monitoring and management of operations throughout the food production facility. It should be appreciated, however, that embodiments are not limited to any particular type of collected data and standards, as any suitable data and standards may be used as a basis for determining compliance.

Distributed work environments, such as food production facilities, may have a large number of interrelating workers and machines. Advances in sensing and communication technology have enabled a variety of different types of information to be collected. However, it may be a challenge to transform the huge amounts of data into effective decision-making, especially when some data may be incomplete or have errors. Furthermore, in some embodiments, acquiring measurements may be costly. Energy is often a limited resource and may be consumed by communication, sensing, and computation. Measurements may not be equally useful and/or may incur different resource expenditures. In some embodiments, a sensor management algorithm may determine which sensors to activate at each time to achieve a desired trade-off between management performance and communication cost.

The sensors may be configured to collect different types of data related to the food production facility, such as human behavioral data, biological data, environmental data, and/or machine data. It should be appreciated that embodiments are not limited in the type of sensors used, as different types of data collected by different types of sensors may be correlated and analyzed by the system. The analysis may be performed by a rules engine, which implements one or more algorithms that analyze the collected data according to the specified instructions and/or standards to detect non-compliance. In some embodiments, reporting devices may be carried by workers and/or supervisors and may provide real time alerts, recommendations, and/or instructions based on the analysis by the rules engine.

FIG. 1 is a schematic illustration of an example of an automated pest trap monitoring and reporting system, according to some embodiments. In some embodiments, the system 100 may be used to monitor and analyze data collected from different sensors distributed throughout a food production facility, and report results of the analysis to one or more reporting devices. Though, it should be appreciated that embodiments are not limited to any particular number of sensors nor the use of reporting devices.

In some embodiments, system 100 may comprise a server 102 that implements a rules engine 104 and a data store 106 that stores collected data and/or predefined specifications. In some embodiments, the server 102 may be a centralized server that aggregates and processes all the aggregated data, although it should be appreciated that embodiments are not limited to a single centralized server, and may implement the rules engine 104 and the data store 106 in a plurality of computing devices that may be distributed throughout the system 100.

Regardless of how the server 102 is implemented, it may be connected to one or more devices that route and/or forward information to or from the server 102. As non-limiting examples, FIG. 1 illustrates a wireless access point 108a and a router 108b that connects the server 102 with a plurality of sensors, for example, sensors 110a, 110b, and 112. The sensors 110a, 110b, and 112 may be any suitable type of sensors that are adapted to collect data from their environments. Though three sensors are shown in FIG. 1, it should be appreciated that the exact number and types of sensors is not limiting.

In some embodiments, one or more sensors, such as trap sensors 110a and 110b, may be configured to collect data related to pest traps. The trap sensors 110a and 110b may be any suitable type of sensor configured to monitor traps. For example, trap sensors may be tactile sensors or remote sensors. Tactile trap sensors may use any suitable actuated mechanism to detect mechanical activation of a trap. Remote trap sensors may include, as examples, infrared sensors or cameras that detect some indication of pests in a trap, such as visible or audible indications. Remote sensors may be placed in any suitable location of an egg farm, to monitor one or more traps.

Alternatively or additionally, some sensors, such as secondary sensor 112 in FIG. 2, may be configured to collect data not directly related to traps. For example, secondary sensor 112 may be, in some embodiments, an environmental sensor that collects data related to ambient conditions around the traps. As another example, secondary sensor 112 may be a sensor that monitors the hens themselves, either via sensors attached to the hens or remote from the hens. As yet another example, secondary sensor 112 may be a human data sensor that detects data related to human workers, who may be responsible for monitoring traps.

By monitoring and analyzing the behavior of human workers responsible for monitoring and inspecting traps, this may improve the accuracy and reliability of monitoring traps. Human sensors may be used to monitor human workers. For example, human sensors may be wearable, such as modified ID badges or modified personal digital assistants (PDAs). Sensors monitoring human workers may use any suitable technology, including, but not limited to, Radio Frequency Identification (RFID) tags, Global Positioning System (GPS) chips, microphones, cameras, accelerometers, to detect any physical human behavior relevant to monitoring traps.

In some embodiments, the sensors 110a, 110b, and/or 112 may be coordinated with other systems in the environment. For example, in the context of an egg farm, one or more sensors may be coordinate with systems configured to control and/or facilitate egg-laying by hens. In some embodiments, an egg farm may utilize an automated illumination system configured to illuminate only certain parts of an egg farm at certain times, and thus regulate the times at which different hens lay eggs. In such scenarios, the sensors 110a, 110b, and 112 may be configured to collect data in coordination with the illumination system.

As a non-limiting example, an image sensor may be configured to only collect images of a trap when the area around the trap is illuminated by the automated illumination system. In some embodiments, sensors may only be activated to collect data when there is no illumination around a trap, if some pests are typically attracted to darkness. It should be appreciated, however, that embodiments are not limited to coordinating sensors with illumination systems, as any automated system may be coordinated with activation or operation of sensors.

Regardless of the exact nature of coordination between sensors and other automated systems in the egg farm, such a coordinated system may have numerous advantages, including but not limited to, conserving power expenditure of sensors by only activating them when useful, and/or improve the accuracy of sensors by only activating them when conditions are suitable for reliable data collection.

It should be appreciated, however, that embodiments are not limited to a particular nature of data collected nor sensors used, as the system 100 may utilize, monitor, and analyze any suitable data relevant to pest infestation in an egg farm. In some embodiments, the sensors 110a, 110b, and 112 may be distributed in different geographic locations in the egg farm, or may be within a common geographic location and/or monitor the same trap or group of traps.

In some embodiments, in addition to collecting data, sensors may perform processing on data collected and/or instructions received. For example, in some embodiments, sensors may perform compression on data that is collected, using techniques in compressive sensing. Such compression may enable a more compact representation of the collected data to be transmitted, thus conserving communication resources. Additionally or alternatively, compression may be performed by intermediate devices, such as a wireless access point (WAP) 108a and/or router 108b. It should be appreciated, though, that embodiments are not limited to compressive sensing, and that data may be transmitted from the sensors 110a-110c to the server 102 in the same form in which they are sensed.

In some embodiments, one or more sensors may communicate with each other. The example in FIG. 1 illustrates trap sensor 110a and secondary sensor 112 communicating via communication link 114, which may be any suitable communication medium, such as wired or wireless. The information transmitted between the sensors 110a and 112 may be, for example, related to the data collected by the sensors and/or may be related to specifications sent from the server 102. In some embodiments, inter-sensor links may be used to relay information from one sensor to another, for example, to perform peer-to-peer routing between sensors that may not otherwise be directly connected to any other access point to the server 102. Additionally, or alternatively, the communication link 114 may be used to enable cooperation between sensors 110a and 112 to help improve the accuracy of data collection, for example, by cross-correlating data collected and verifying consistency.

In some embodiments, the sensors may be specifically configured to collect data that is most relevant to determining the location(s) and/or number of pests in an egg farm. In some embodiments, the sensors may be dynamically adjusted in real time based on the collected and analyzed data. For example, a particular sensor may be adapted to collect more and/or different data when a potentially trapped pest is detected in the data collected by that sensor. In some embodiments, such adjustments may be made by the server 102, or by any other computing device that has access to the data collected by the sensor. In some embodiments, sensors may also have an input/output interface, such as a keyboard or a screen, to enable manual control of the sensor.

Regardless of the exact nature of the sensors 110a, 110b, and 112, and the techniques by which they communicate with the server 102 and/or each other, the sensors 110a, 110b, and 112 may collect and transmit data to the server 102 for analysis using the rules engine 104 and storage in the data store 106. The server 102 may be configured to recognize data collected from different sensors, and analyze the different types of data using the appropriate specifications applied by the rules engine 104. As such, in some embodiments, the system 100 may be able to monitor and analyze traps that are located throughout an egg farm.

In some embodiments, the collected data may be stored in a computer memory, such as a data store 106. The data store 106 may be integrated with the server 102 or may comprise multiple memory locations distributed in different parts of a network. The stored data may include any of the data described above, or any other suitable type of data collected by sensors and/or relevant to processing the data collected by sensors. In some embodiments, the data store 106 may store data, either historical or statistical, for individual traps or an aggregate group of traps. Specifications may include, as a non-limiting example, standards established by a suitable entity, such as the government, industry, or a company. In some embodiments, the data store 106 may be accessible by one or more other computing devices, such as by the sensors 110a, 110b, and 112 or other types of devices.

In some embodiments, the system 100 may implement the rules engine 104 configured to aggregate and analyze the different types of collected data and determine an appropriate course of action. In some embodiments, the rules engine 104 may be able to learn and make decisions in real time. As a non-limiting example, the rules engine 104 may analyze trap data and/or other types of collected data, such as human behavioral data, and estimate a likelihood of trapped pests. The server 102 may then determine whether or not to assign a worker to manually inspect traps(s).

In some embodiments, the system may predict future conditions of the system related to monitoring pest infestation, such as likelihood of pest infestation in a particular area of the egg farm and/or likelihood that workers will accurately inspect and report traps in particular areas of an egg farm. The system may then proactively recommend that more traps be placed in, and/or follow-up inspections be performed for, certain areas of the egg farm. Such estimations and/or predictions may be made, for example, by a suitable machine learning algorithms trained with past historical data from the traps, environment around the traps, and/or human behavior.

Regardless of the exact nature of the algorithms implemented by the rules engine 104, the rules engine 104 may be able to determine a desired plan of action based on the different types of data collected by the sensors 110a, 110b, and 112. Determining a desired plan of action may be based on any suitable technique. As non-limiting examples, the rules engine 104 may perform linear/nonlinear optimization algorithms, dynamic programming, and/or Monte Carlo simulations to select one or more actions that should be performed to ensure proper detection and removal of trapped pests.

In some embodiments, the rules engine 104 may be able to cross-correlate different types of data collected by different sensors 110a, 110b, and 112, some of which may be related to a particular trap or area of traps. Some of the sensors 110a, 110b, or 112 may be located at a common location, or may be distributed at different locations. Regardless of the exact location of the sensors, the rules engine 104 may be able to integrate the different types of data collected by the sensors 110a, 110b, and 112 to detect non-compliance and/or inconsistencies related to monitoring and reporting pest infestation. For example, if trap sensors 110a, 110b monitor traps and another sensor 112 monitors hen behavior, then the rules engine 104 may be able to correlate trap sensor data with hen behavioral data to determine a likelihood that pests are present in a location. In some embodiments, based on the analysis of the collected data, the rules engine 104 may be able to dynamically reconfigure one or more sensors 110a, 110b, or 112, for example, to collect more detailed or different types of data.

Regardless of the exact nature of the rules engine 104, different types of collected data may be analyzed and correlated with a set of specifications to determine non-compliance and/or potential non-compliance, and to provide re-mediation instructions and/or recommendations for future action. In some embodiments, compliance may be related to the sensor data complying with a set of prescribed rules and/or instructions. These rules and/or instructions may be provided by any suitable entity, such as an entity in a government, industry, or a company. Alternatively or additionally, the rules and/or instructions may be provided by the system itself, in response to previous analysis performed on sensor data. Regardless of the exact nature of the compliance, the system may analyze various types of sensor data related to pest infestation and determine a compliance status.

The results of the analysis may be provided to one or more devices, such as reporting devices 116a, 116b, 116c. Although three such reporting devices are illustrated in FIG. 1, it should be appreciated that embodiments are not limited to any particular number of reporting devices, and that the use of reporting devices is optional. In some embodiments, the results of the analysis by the rules engine 104 may be provided back to the sensors 110a, 110b, and 112, which may have a display or other output mechanism to provide information about the results of the rules engine 104 to an appropriate operator or supervisor.

In some embodiments, if reporting devices 116a-116c are used, then such reporting devices may be any suitable device configured to display information related to the analysis of the rules engine 104. For example, in some embodiments, reporting devices 116a-116c may include mobile devices, personal computers, or workstations. The reporting devices 116a-116c may be specially designed devices, or may be unmodified consumer devices, such as smartphones with downloaded applications, configured to display the results of the rules engine 104. In some embodiments, the reporting devices 116a-116c may have a dashboard display that allows a user to interact with the reporting devices 116a-116c.

For example, the reporting devices 116a-116c may enable a user to provide feedback to the server 102 based on results of the rules engine 104. Such feedback may include, for example, specific actions or instructions that should be taken by one or more workers and/or machines, and/or requests for more data or different types of data to be collected by the sensors 110a, 110b, or 112. In some embodiments, the reporting devices 116a-116c may enable a user to input new or updated specifications to be applied by the rules engine 104.

Regardless of the exact nature or use of the reporting devices 116a-116c, a user operating such reporting devices may be provided with real time information regarding pests present in a trap or group of traps. As such, the system 100 may provide an integrated real time monitoring and management capability for an egg farm, in which problems may be detected and mitigated proactively, potentially before they propagate to other parts of the egg farm. The heterogeneous nature of different sensors involved in the system 100 may be seamlessly integrated by the rules engine 104, which may be aware of the different relationships between the data and, in some embodiments, is able to learn behavior and trends of pest infestation, to accurately predict potential sources of non-compliance before such problems manifest or grow.

FIG. 2 illustrates one possible example of an environment 200 in which system 100 may be used. In some embodiments, the environment 200 may be an egg farm in which sensors are configured to monitor one or more traps placed at particular locations in or around the egg farm. For example, the egg farm may have hens housed in rows of battery cages or allowed to roam in a free-range barn or enclosure. Traps may be placed near battery cages, nest boxes, feed/water storage, or at any other appropriate location where pests could be present. It should be appreciated, however, that an egg farm is merely one example of environment 200, as environment 200 may represent any suitable environment in which sensors may be used to remotely monitor and determine the location of potential pest infestation.

In some embodiments, a centralized server 102 may monitor and analyze data collected from one or more sensors. Though, it should be appreciated that embodiments are not limited to a single centralized server and may utilize multiple computing devices to monitor and analyze data. Regardless of the exact number and nature of computing devices that analyze and monitor data, a rules engine 104 and a data store 106 may be used to analyze and store various data collected throughout the egg farm, and to monitor compliance with one or more specifications and/or instructions related to pest infestation and/or trap monitoring.

In the example of FIG. 2, three traps are illustrated, rodent traps 202a, 202b, and 202c, which may be placed in any suitable location in the environment 200. It should be appreciated, however, that embodiments are not limited to trapping rodents, as traps for any suitable pest may be monitored and reported. In the example of FIG. 2, the rodent traps 202a-202c may be any suitable rodent traps, either single-capture devices or cumulative-catch devices, using any suitable rodent trapping technology as is known in the art. It should be appreciated that embodiments are not limited to a particular type of rodent trapping system, as any suitable trap or trapping system for capturing rodents may be used.

In some embodiments, a rodent trap may have a closing mechanism for enclosing a rodent within the trap, such as door 204 in trap 202b. This closing mechanism may be activated by any suitable technique as is known in the art, such as via mechanical actuation, electronic activation, or any other technique that enables the trap to enclose rodent(s) to dissuade escape from a trap. As an example, the door 204 may be configured to drop-down vertically from a top of the trap 202b, as is illustrated in FIG. 2, although it should be appreciated that embodiments are not limited to any particular configuration of how the door 204 closes on a trap, as the door 204 may be configured to close horizontally, in a folding manner, or any other direction and methodology.

In some embodiments, one or more traps may have a viewing mechanism that allows external view into the trap, such as window 206 in trap 202c. Such a viewing mechanism may, for example, assist a human inspector or remote camera to determine whether there is a rodent trapped inside the trap. It should be appreciated, however, that a viewing mechanism may be used for any purpose, not limited to visual inspection of rodents inside the trap. As non-limiting examples, the window may allow for viewing of a visual indicator inside the trap, such as a light or other mechanism for determining the presence of rodents inside a trap.

In some embodiments, an indicator may be placed outside of a trap, such as indicator 208 attached to trap 202c in FIG. 2. As a non-limiting example, the indicator 208 may be a visual indicator, such as a low-voltage light emitting diode (LED) configured to emit a light to indicate the presence of rodents in the trap 202c. The indicator 208 may be powered by any suitable technique, such as battery, RFID, solar, mechanical transduction, or any other technique for powering low-voltage devices known in the art, as embodiments are not limited in this regard. It should be appreciated, however, that embodiments are not limited to using visual indicators, as indicator 208 may use audible, tactile, or any other suitable technique to indicate the presence of rodents in trap 202c.

In some embodiments, traps may be monitored by one or more sensors, such as sensors 210a, 210b, and 210c. Sensors of any appropriate type or number may be used, as embodiments are not limited in this regard. For example, a sensor may detect the activation of a trap, such as closing of a trap door or any other suitable technique of determining that a trap has been activated. In FIG. 2, for example, sensor 210b is configured to detect the activation of a door 204 on trap 202b. A sensor may use any suitable technique to detect the activation of a door, such as mechanical detection (a switch and/or trigger coupled to an actuator, transistor, etc.), vibration (triboelectric, seismic, and inertia-switch sensors), or other suitable technology configured to detect activation of a trap door.

Additionally or alternatively, a sensor may detect the weight of a trap to determine a number of rodent in the trap. A weight-based sensor may provide information in addition or as an alternative to other types of trap sensors. For example, in some embodiments, if a weight-based sensor is used in conjunction with an activation sensor, such as sensor 210b, then the additional weight-based sensor information may enable detecting multiple rats entering in a single activation of a trap door, or determining whether a detected trap activation was caused by a false alarm.

In FIG. 2, for example, sensor 210a is configured to detect the weight of trap 202a. This may be accomplished by any suitable technique, such as a weight scale 218 placed underneath the trap 202a. It should be appreciated, however, that embodiments are not limited to using a weight scale underneath the trap, as any suitable technique may be used for determining the weight of a trap.

In some embodiments, sensors may be configured to use remote detection of traps. In FIG. 2, for example, sensor 210c is configured to remotely detect the present of rodents in trap 202c. The sensor 210c may use any suitable technique to remotely monitor the trap 202c. For example, the sensor 210c may have a camera (stationary or mobile) that takes images outside and/or inside the trap 202c. In some embodiments, one or more image processors may perform pattern recognition and/or machine vision to determine, from the images, activation of a trap and/or number of rodents in a trap.

For example, an image processing algorithm may compare images inside/outside the trap with a reference image. The reference image may correspond to a trap with an open/closed door, or a trap with one or more rodents inside a viewing window, or any other suitable reference image that indicates presence or absence of rodents in a trap. In the setting of an egg farm, sensor 210c may be coordinated with other systems in the egg farm such as a timed illumination system configured to induce and control egg-laying behavior in hens.

It should be appreciated, however, that remote sensors such as sensor 210c are not limited to using cameras, as remote sensors may use any suitable remote detection mechanism. For example, a remote sensor may use an infrared (IR) mechanism to remotely detect temperature and/or motion outside/inside the trap 202c. As yet another example, the remote sensor 210c may use reflection of transmitted energy (laser radar, ultrasonic, microwave radar) to detect pests inside a trap.

Sensors may be powered by any suitable powering technique, including but not limited to, battery, photovoltaic, vibrational, temperature gradient, electromagnetic, etc. As another example, if a sensor is electronically attached to a trap, then the sensor may be powered by motion of the trap, such as a trap door or movement of rodents inside the trap.

Regardless of the exact nature of the sensors 210a-210c, the sensors may be configured to collect any suitable information related to the presence or number of rodents in traps, according to any suitable technique. In some embodiments, the sensors may be simplified sensors that each collect only minimal information and processing may be performed on the collected data. Such processing may be performed, for example, by the sensors themselves or by other computing devices, such as by intermediate routes or by the server 102.

In some embodiments, one or more other sensors, such as secondary sensor 212, may be used in addition or as an alternative to trap sensors 210a-210c. The secondary sensor 212 may be configured to provide additional data to assist the rules engine 104 in determining the presence and/or location of pest infestation. Such data may be used by itself to determine the presence of rodents, or may be used in addition to data collected by trap sensors to provide redundancy and improve accuracy.

As non-limiting examples, the secondary sensor 212 may detect information related to hens (vital signs, hen sounds and motion, etc.), related to the environment around the hens or traps (temperature, light, sound, etc.), and/or related to human workers assigned to inspect traps or areas around the traps. Such data, while not necessarily directly related to the traps themselves, may be used in conjunction with other types of sensor data to improve the reliability of detecting pest infestation.

If the secondary sensor 212 is configured to detect behavior of humans, then the monitored behavior of humans may include, for example, time spent on certain tasks, completion of tasks, and/or efficiency in completing tasks, related to monitoring rodent traps. Such human behavioral data may be correlated with other types of data collected within the environment 200 and analyzed in aggregate by the server 102 to determine an overall rodent infestation status.

As a non-limiting example, in some embodiments, hen sensors, environmental sensors, and human sensors may be used in conjunction with trap sensors. In such scenarios, even if a trap sensor does not indicate the presence of pests, if hen sensors indicate a disturbance among a particular group of hens while environmental sensors indicate no abnormalities in the environment around that group of hens and human sensors indicate that human workers have not recently inspected that area, then the rules engine may determine that a worker should be assigned priority to inspect traps surrounding that group of hens, to check for potential pest infestation that could be causing the disturbance in the group of hens.

As such, different types of sensors and/or different types of information collected by sensors may be used in conjunction to provide redundancy and/or improve the accuracy of detecting whether or not a trap has a rodent. Improving the accuracy of trap detection may, for example, not only improve detection of rodents trapped inside traps, but may also reduce the probability of false alarms. Reducing false alarms may help reduce the number of unnecessary assignments of human workers to physically inspect empty traps located in potentially unsafe or unsanitary conditions. As such, improved accuracy of trap detection may enable improved efficiency, productivity, and safety in assigning tasks to a work force in a large distributed work environment.

In some embodiments, in addition to collecting data, sensors may perform processing on data collected and/or instructions received. For example, in some embodiments, sensors may perform compression on data that is collected, using techniques in compressive sensing. Such compression may enable a more compact representation of the collected data to be transmitted, thus conserving communication resources. It should be appreciated, though, that embodiments are not limited to compressive sensing, and that data may be transmitted from the sensors 210a-210c and 212 to the server 102 in the same form in which they are sensed.

Sensors 210a-210c and 212 may collect as much or as little data as often or as infrequently as desired. For example, data may be collected for every individual trap or in aggregate for groups of traps. Data may be collected hourly, daily, weekly, randomly, or at any suitable times that may or may not be predetermined. In some embodiments, a sensor may transmit data whenever new information detected, or at periodic intervals. To save power, some sensors may transmit information in response to a query/poll from a remote device, such as receivers or server 102.

In some embodiments, the sensors may have wireless transmitters configured to transmit the collected information to one or more remote receivers. It should be appreciated, however, that not all sensors are necessarily required to be configured with wireless transmitters, as sensors may use any suitable technique to enable the collected information to be gathered from the sensors, such as wired transmission or removable memory. In some embodiments, one or more sensors may simply be configured to indicate of the presence of rodents, such as via light or sound, and may not necessarily transmit any separate information at all.

Regardless of the exact function, number, and location of sensors in environment 200, one or more sensors may collect data relevant to rodent infestation, and transmit that collected data to one or more computing devices, such as the server 102. Sensors may transmit trap information or other related information to one or more receivers, either directly or relayed via other sensors and/or devices.

Sensors may transmit the information by any suitable technique. For example, in some embodiments, sensors may transmit data via receivers 214a, 214b, and 214c. Such receivers may be part of relaying computing devices such as, for example, wireless access points, routers, or any other suitable computing device that is able to receive and transmit information. In some embodiments, a receiver may be part of a sensor, and may be configured to relay information from other sensors to the central server 102 or to other computing devices or receivers.

The transmission from sensors may be in any suitable format. As non-limiting examples, binary information may be transmitted, indicating the presence or absence of detected rodents in a trap, or more detailed non-binary information may be transmitted to indicated a number of rodents, location of the trap, or other suitable information. It should be appreciated, however, that embodiments are not limited to any particular format or representation of data, as the information collected by sensors may be transmitted in any suitable representation. For example, in some embodiments, a sensor may transmit information in multiple stages, for example, first transmitting simpler (e.g., binary) information, then transmitting more detailed information, either upon request or on its own.

Further, any suitable signaling technique may be used when transmitting data from the sensors. For example, a signaling technique may be based on amplitude modulation or frequency/phase modulation, etc., to convey the information. Regardless of the exact nature of a signaling technique used, data may be transmitted from one or more sensors to one or more receivers, such as receivers 214a-214c.

In some embodiments, transmissions from multiple sensors and/or other computing devices to a receiver may be coordinated with an access scheme. Examples include, but are not limited to statistical multiplexing (e.g., carrier sense multiple access (CSMA)) and orthogonal multiplexing (each sensor may have a unique signal, and the system may map the signal to a known location). Some examples of orthogonal multiplexing include: frequency division multiplexing (FDM), in which every sensor is assigned a unique frequency; time division multiplexing (TDM) in which every sensor is assigned a unique time slot; code division multiplexing (CDM), in which every sensor is assigned a unique code; and space division multiplexing (SDM), in which multiple antennas with beam-forming are used. It should be appreciated, however, that any suitable transmission and modulation technique may be used in the environment 200 to coordinate transmission from multiple sensors and/or relays, as embodiments are not limited in this regard.

In some embodiments, results of the analysis by server 102 may be displayed on a device 216, which may be a mobile device operated by a user. As non-limiting examples, the device 216 may present alerts regarding compliance or potential non-compliance, or may present instructions and/or recommendations based on analyzed data. The instructions and/or recommendations may be based on a set of protocols established by an entity, such as the egg farm itself, a governmental body, an industry organization, or a customer. The instructions and/or recommendations may relate to operation of machines, handling of traps, recording or reporting certain actions, or any other task related to managing the inspection of traps and pest infestation. Additionally or alternatively, the mobile device 216 may include a sensor that is configured to detect data from a human, using for example, microphones and/or other sensors.

The rules engine 204 may recognize an inconsistency between various types of collected data, and may generate an alert indicating a potential pest infestation. Such an alert may be used, for example, by an egg farm to check the indicated trap(s) location(s). As such, the server 102 may be able to analyze the collected data to detect potential sources of non-compliance, even when other sources of data, whether collected by sensors or entered by humans, do not indicate any problems. The rules engine 104 may also be configured to detect lack of collected data, whether due to malfunctioning sensors or due to human error and/or fraud, and to generate alerts based on the lack of collected data. Algorithm/rules can aggregate these different types of information to infer pest infestation.

In some embodiments, such cross-correlation of different types of data may also reduce occurrences of false alarms, in which non-infested areas are mistakenly detected to be infested. Such false alarms may degrade efficiency of an egg farm, by causing the egg farm to implement various actions to check on the status of traps, which may otherwise not be necessary. For example, in some embodiments, this may reduce the need to send humans into potentially unsafe conditions within a large egg farm to check on the status of areas that are infestation-free.

In some embodiments, the system may be able to provide such real-time monitoring of traps, using various sensor data. Such a system may enable not only faster response and locating and handling pest infestation, but may also enable proactive actions to mitigate conditions that may potentially cause areas to become infested. In some embodiments, this may be achieved by cross-correlating data collected from different types of sensors, and detecting any inconsistencies or anomalies that may indicate non-compliance with a set of provided standards. Such a preventative system, may, in some embodiments, drastically improve the efficiency and safety of an egg farm, and particularly those that monitor hundreds or thousands of traps distributed over a large egg farm.

The system, in some embodiments, may also have the ability to adaptively learn and predict future states of the system and/or behavior of human workers to facilitate proactive alerts and improve efficiency of operations. Such learning and predictive analysis may be enabled, in some embodiments, by any suitable learning technique, such as machine learning algorithms, neural networks, simulations, or other suitable techniques, as embodiments are not limited in this regard. Regardless of the exact nature of the analysis implemented by the rules engine 104, the analysis may be configured to operate on a wide variety of data collected by different sensors, and in some embodiments, stored in the data store 106. The data store 106 may comprise data that is collected from sensors, and also may comprise standards, regulations, and specifications that should be followed by one or more entities and the distributed work environment.

FIG. 3 illustrates one example of a data store 300 that stores various types of data and standards. It should be appreciated, however, that embodiments are not limited to storing these particular types of data, as more or less types of data and standards may be stored suitable to the environment in which the system operates.

Although embodiments are not limited to the exact nature or number of types of data stored in the data store 300, in some embodiments, the data store 300 may store data related to individual traps, a group of traps, and/or a region of traps. For example, trap data may be stored in a trap database 302, in which data for individual traps, groups of traps, or regions of traps may be stored in one or more data tables, such as 304a, 304b, and 304c. In some embodiments, a data table may comprise one or types of data related to potential pest infestation for the trap(s). For example, trap table 304a may store at least one or more of activation data 306 related to activation(s) of the trap(s), weight data 308 related to weight(s) of the trap(s), and location data 310 related to location(s) of the trap(s). In some embodiments, one or more types of data stored for a trap or groups of traps may include historical data, such as data collected over a past period of time.

It should be appreciated, however, that embodiments are not limited to these particular types of data, as data store 300 may store more or less data related to traps. For example, in some embodiments, data store 300 may include data collected from various types of sensors, such as images, sounds, biological data (collected from sensors attached to the hens or remote from the hens), environmental data (representing ambient conditions in or around the egg farms), etc. Further, it should be appreciated that embodiments are not limited to any particular number of traps or groups of traps for which data is stored, nor a particular number of data tables.

In some embodiments, the data store 300 may store one or more specifications, standards and/or instructions related to analyzing data that has been collected by the sensors. For example, the data store 300 may comprise a rules/specifications database 312, which may store data related to various standards, such as regulations, rules, and/or instructions applicable to the egg farm. As non-limiting examples, the rules database 312 may comprise governmental regulations 314, which may represent international, federal, state, and/or local regulations. In some embodiments, rules database 312 may comprise industry standards 316, which may represent protocols and/or standards established by, for example, industry organizations or trade groups. In some embodiments, the standards database 312 may comprise company specifications 318, which may represent company-specific protocols and/or rules established by the egg farm and/or other entities such as customers or partners of the egg farm.

It should be appreciated, however, that the rules database 312 is not limited to these specific types of rules, and that more or less rules may be stored in the data store 300. For example, in some embodiments, there may be no applicable governmental regulations 314 and/or no applicable industry standards 316, in which case the standards database 312 may only comprise company specifications 318.

The data store 300 may also comprise, in some embodiments, instructions that have been issued by the system or by human management. Such instructions may be stored in any suitable location in the data store 300, for example in rules database 312, or in the trap database 302 associated with a particular trap, or in a separate database altogether.

In some embodiments, data store 300 may store data related to human behavior and actions. For example, the data store 300 may include a human database 322 that stores data for each worker or groups of workers, such as in worker table 324. Such data may include, for example, historical performance data for particular workers, specific tasks assigned to workers, and/or any suitable data related to monitoring and reporting pest infestation in an egg farm.

FIGS. 4 and 5 are flow charts that describe examples of processing that may be performed by a server (e.g., server 102 in FIG. 2), or any other computing device that analyzes data collected from sensors. The various steps involved in FIGS. 4 and 5 may be performed in real time as data is collected and received from the sensors, or may be performed in an offline manner with data already available for analysis. Regardless of the exact times and manner in which the steps of FIGS. 4 and 5 are implemented, the processes described in these examples may be used to analyze and aggregate data collected from sensors, estimate a status of pest infestation. The server 102 may also predict a future status of infestation, detect non-compliance or potential non-compliance, and/or generate alerts instructions based on the analysis.

Based on sensor data, in some embodiments, it may be desirable to improve sensitivity (detect trapped pests) while minimizing false alarms (reduce manpower to inspect empty traps). To achieve a desired tradeoff, the rules engine may be tuned to analyze one or more types of data collected by one or more types of sensors, to formulate an aggregate opinion of whether and where to inspect for trapped pests. The rules engine may perform its analysis based on historical data or statistical averages.

For example, the rules engine may use historical data collected from sensors to analyze and/or predict potential pest infestation. For example, the rules engine may analyze a past window of data collected from one or more sensors, or sampled data from the past history, or any suitable set of past historical data. The data may be any type of data collected by any type of sensor, such as trap sensors, human behavioral sensors, environmental sensors, etc. In some embodiments, the rules engine may utilize machine learning algorithms to predict potential non-compliance, such as potential failure by a human worker to inspect certain traps, or to perform other actions related to trap monitoring. The rules engine may generate an alert if there is a deviation from any acceptable standard.

In some embodiments, the rules engine may use statistical parameters such as mean or variance, compare collected data to those statistical parameters, and generate an alert if the data is outside of a standard deviation of the mean. The statistical data may be related to traps, such as weight of a trap or image data captured for a trap, etc., or may be related to non-trap data such as human behavioral data, environmental data, hen behavioral data, etc. Other statistical-based methods may be used for detecting deviation from normal behavior, such as those based on a priori statistical (Bayesian) models, though it should be appreciated that embodiments are not necessarily limited to using statistical models, or even statistical data at all.

Based on the analysis and/or prediction, the rules engine may generate alerts and/or instructions to a user regarding at least one potential location of trapped pests.

FIG. 4 is a flowchart of an example of a process 400 that may be implemented by a server (e.g., server 102 in FIGS. 1 and 2). Process 400 may begin in block 402 with the server accessing data and/or standards from a data store (e.g., data store 300 in FIG. 3). Though, it should be appreciated that data and/or standards may be accessed from any suitable data store, which may be local to the server or at a remote location, for example, connected to a network accessible by the server. In some embodiments, the data and/or standards that are accessed in block 402 may be a subset of the data and standards stored in a data store. In scenarios in which there is a lame amount of collected data and/or standards, such selective accessing of information from the data store may enable more efficient and faster analysis. For example, different types of data may contribute different amounts of utility to an analysis of compliance with one or more standards. The rules engine may be able to determine, based on prior measurements and analysis, which types of data yields the highest expected information gain, and may access only those data.

In some embodiments, sensors may be configured to collect or not collect certain types of data. For example, some sensors may be configured not to collect data in order to conserve energy and/or communication resources, based on a determination that data collected by those sensors would yield smaller expected information gain than other sensors. Regardless of the exact nature in which data is accessed and/or available, the system may recognize that only a subset of data that could potentially be collected by the sensors may be sufficient to yield a desired level of estimation and/or prediction accuracy, and that data collected by other sensors may yield diminishing returns.

Based on the data and standards that have been accessed from the data store, in block 404, the rules engine may be applied to the collected data and prescribed rules, to determine pest infestation throughout the egg farm. In block 406, if it is determined that the analyzed data indicates pest infestation, then, in block 408, the system may generate one or more alerts. For example, such alerts may be indicated on remote reporting devices (e.g., reporting device 216 in FIG. 2), or on the sensors, or on any suitable computing device. Such alerts may include an indication of location(s) or other information related to traps that potentially contain pests.

Additionally or alternatively, the system may, in block 410, issue specific instructions and/or recommendations regarding handling of traps that potentially contain pests, for example, on a reporting device (e.g., reporting device 216 in FIG. 2). For example, block 410 may involve issuing instructions to adjust or modify human tasks and/or machine operations to handle pest infestation, though embodiments are not limited in this regard.

In some embodiments, even if the rules engine has not detected any non-compliance based on the collected data, the system may still issue any necessary instructions or recommendations in block 410 after pest detection in block 406, without generating any alerts.

After appropriate determination and issuance of instructions have been performed, in some embodiments, in block 412, the data store may be updated with results of the analysis and/or the issued instructions. The data store may also be updated with revised standards and/or predicted data based on results of the analysis.

It should be appreciated that issuing instructions in block 410 and updating the data store in block 412 are optional, and in some embodiments, an alert and location of pest infestation may be generated, in case of detected or predicted infestation, without any specific instructions or updates of the data store.

FIG. 5 is a flowchart of an exemplary process 500 of processing by a rules engine. For example, in some embodiments, process 500 may represent details of the processing performed by the rules engine (e.g. block 404 of FIG. 4) to analyze the collected data. The process 500 performed by a rules engine may apply any combination of suitable techniques to analyze the different types of data collected by sensors, to detect non-compliance related to monitoring pest infestation, predict potential future non-compliance, and/or determine the appropriate instructions based on the analysis. Although process 500 in FIG. 5 illustrates one possible sequence of processing that may be performed by the rules engine, it should be appreciated that embodiments are not limited to any particular sequence or nature of processing and, in general, the rules engine may apply any suitable processing to the collected data to determine pest infestation.

In block 502, the rules engine may correlate various types of collected data, which, in some embodiments, may comprise the different types data described above in relation to FIG. 3. Though, it should be appreciated that in some embodiments, more or less data may be used. In some embodiments, if the data was compressed by the sensors prior to communication, then in step 502, the received data may be decompressed before performing correlation. Additionally or alternatively, decompression of any compressed data may be performed in block 402 of FIG. 4.

In some embodiments, correlation of the data may comprise performing data fusion and/or data mining to extract information that may be relevant from within the data. For example, in some embodiments, data fusion may comprise processing the data collected by the sensors to create a more compact representation of information relevant to determine non-compliance related to pest infestation. In some embodiments, block 502 may, additionally or alternatively, apply data mining algorithms, which may comprise detecting any anomalies, patterns, classifications, and/or other associations between the different types of data collected. In some embodiments, if the collected data is voluminous, then the data fusion and/or data mining algorithm may enable representing the voluminous data in a more compact manner. Though, it should be appreciated that block 502 is not necessarily limited to generating compact representations of the collected data, as correlation of data may comprise any suitable processing to determine correlations between the data collected by the different types of sensors.

If the data that is correlated in block 502 is insufficient to determine non-compliance in pest infestation, then in block 504, it may be determined that more data is necessary. Then, in block 506, more data may be obtained, either from the data store or from the sensors, and the updated data may be used to perform the correlation in block 502. In some embodiments, the updated data in block 506 may simply be accessed by querying the data store for the desired data, and in some embodiments, a communication may be sent to one or more sensors to collect and transmit more or different types of data. Regardless of how this updated data is obtained, the processing in blocks 502 and 504 may be repeated until it is determined that a sufficient amount of data is available.

Then in block 508, in some embodiments, the rules engine may generate an estimate or prediction of non-compliance, based on the measured data and any correlation performed in block 502. The estimation and/or prediction of non-compliance may be achieved by any suitable technique. For example, the rules engine may apply one or more machine learning algorithms. As non-limiting examples, machine learning algorithms may comprise neural networks, linear/non-linear optimizations, Bayesian learning networks, or other suitable techniques that can analyze data measured from a system to predict a future parameter status of the system.

For example, if a Kalman filter is used, then the predictive step of the Kalman filtering processing may be used to generate an estimate of a current compliance status, or a prediction of a future compliance status, based on past estimates of compliance status and measurements from the sensors. As a non-limiting example, a compliance status for a particular trap or group of traps may be specified as a binary 1 or 0, where a status of 0 indicates that the trap(s) is free of pests and a status of 1 indicates that the trap(s) is infested. Alternatively, non-binary results may be generated, indicating various levels of certainty that a trap is infested.

In some embodiments, the rules engine may be able to generate an estimate or prediction of the binary compliance status on past measurements and estimates of pest infestation status. In addition, in some embodiments, a confidence score may be generated for the prediction, indicating a level of confidence in the prediction. For example, the confidence score may be a maximum a posteriori probability (MAP), though embodiments are not limited in the use or nature of a confidence score.

In some embodiments, if predictive processing is used by the rules engine, then it may enable proactive monitoring and management of pest infestation. As such, even if a current estimate by the rules engine, other determinations by other means, do not indicate existing pest infestation, the rules engine may be able to use predictive analysis to proactively determine whether certain areas of an egg farm may potentially be likely for pest infestation. Regardless of the exact nature of estimation and/or prediction performed in block 508, any suitable machine learning algorithm may be used, whether supervised with actual measurements from traps, or unsupervised with only data collected from sensors external to the traps, to generates estimates and/or predictions of pest infestation.

In block 508, the determined estimates and/or predictions of the status of pest infestation may be correlated with a prescribed set of rules to determine whether action should be taken. For example, a standard, specification or instruction may require that three consecutive pest infestation indications (e.g., a binary indicator of 1) for a trap or group of traps requires action to be taken. As another example, if confidence scores are used, then determination of compliance may be based on a particular threshold of confidence score above which a decision is to be made. The rules (e.g., from standards database 312 in FIG. 3) applied in block 508 may be any suitable set of rules provided by governmental agencies, industry trade groups, or a specific company.

In some embodiments, based on the analysis of the data collected by the sensors, in block 510, the rules engine may determine appropriate actions to be taken. For example, actions may involve manually checking a trap or group of traps, adjusting a machine setting on a trap or sensor, and/or double-checking environmental or biological data detected to be anomalous. In some embodiments, such actions may be performed in response to a detected pest infestation and/or potential future pest infestation, or may be performed even when no pest infestation is detected/predicted.

In some embodiments, block 510 may additionally or alternatively comprise modifying or reconfiguring sensors, to collect more, less, or different types of data. For example, the rules engine may determine, based on the results of the analysis, which collected data are most useful in determining infestation in a particular trap or group of traps. Based on such determination, the system may reconfigure the sensors such that only those sensors whose measurements yields the highest expected information gain perform data measurement and communication. In some embodiments, this may enable improved usage of resource constrained sensors, and/or may streamline the processing by the rules engine by correlating only the data that is most useful in block 502. In addition, or as an alternative, to resource management, sensor reconfiguration may be performed to improve the accuracy and reliability of estimation and/or prediction of pest infestation. Such sensory configuration may comprise collecting more data, or different types of data at certain critical control points, or other parts of the egg farm.

It should be appreciated, however, that the system need not necessarily be automatically reconfigured, and may determine existing and/or potential non-compliance without also determining modifications to sensors, human work tasks, or other parts of the system.

Such a trap monitoring system may enable automating the detection, counting, and localization of pest infestation, and reduce the reliance on potentially erroneous and/or fraudulent human inspection.

FIG. 6 illustrates an example of a suitable computing system environment 600 on which the invention may be implemented. This computing system may be representative of a central server (e.g., server 102 in FIG. 1), a sensor (e.g., sensors 110a, 110b, 112 in FIG. 1), or a reporting device (e.g., reporting devices 116a-116c in FIG. 1). However, it should be appreciated that the computing system environment 600 is only one example of a suitable computing environment and is not intended to suggest any limitation as to the scope of use or functionality of the invention. Neither should the computing environment 600 be interpreted as having any dependency or requirement relating to any one or combination of components illustrated in the exemplary operating environment 600.

The invention is operational with numerous other general purpose or special purpose computing system environments or configurations. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 6, an exemplary system for implementing the invention includes a general purpose computing device in the form of a computer 610. Components of computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 610 typically includes a variety of computer readable media. Computer readable media can be any available media that can be accessed by computer 610 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 6 illustrates operating system 634, application programs 635, other program modules 636, and program data 637.

The computer 610 may also include other removable/non-removable, volatile/nonvolatile computer storage media. By way of example only, FIG. 6 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through an non-removable memory interface such as interface 640, and magnetic disk drive 651 and optical disk drive 655 are typically connected to the system bus 621 by a removable memory interface, such as interface 650.

The drives and their associated computer storage media discussed above and illustrated in FIG. 6, provide storage of computer readable instructions, data structures, program modules and other data for the computer 610. In FIG. 6, for example, hard disk drive 641 is illustrated as storing operating system 644, application programs 645, other program modules 646, and program data 647. Note that these components can either be the same as or different from operating system 634, application programs 635, other program modules 636, and program data 637. Operating system 644, application programs 645, other program modules 646, and program data 647 are given different numbers here to illustrate that, at a minimum, they are different copies. A user may enter commands and information into the computer 610 through input devices such as a keyboard 662 and pointing device 661, commonly referred to as a mouse, trackball or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a video interface 690. In addition to the monitor, computers may also include other peripheral output devices such as speakers 697 and printer 696, which may be connected through a output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 6. The logical connections depicted in FIG. 6 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modern 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 6 illustrates remote application programs 685 as residing on memory device 681. It will be appreciated that the network connections shown are exemplary and other means of establishing a communications link between the computers may be used.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. At least one sensing device configured to monitor a pest trap, the at least one sensing device comprising:
a microsensor configured to detect pest infestation information; and
a transmitter configured to transmit the pest infestation information.

2. The at least one sensing device of claim 1, wherein the pest infestation information comprises trap data indicating the presence of a pest in the pest trap.

3. The at least one sensing device of claim 1, wherein the pest infestation information further comprises at least one of human behavioral data, environmental data, biological data, or machine data.

4. The at least one sensing device of claim 1, further comprising a motion sensor that detects motion in or around the pest trap.

5. The at least one sensing device of claim 1, further comprising an imaging device that captures images in or around the pest trap.

6. The at least one sensing device of claim 5, wherein the imaging device comprises an infrared sensor and the at least one property indicates a temperature in or around the pest trap.

7. The at least one sensing device of claim 5, wherein the imaging device comprises an optical imaging device, and the at least one property indicates a physical configuration of the pest trap.

8. The at least one sensing device of claim 7, wherein the optical imaging device is further configured to coordinate the timing of image capturing with at least one illumination device.

9. The at least one sensing device of claim 1, further configured to be periodically polled for updates.

10. A system of monitoring pest traps, the system comprising:
at least one sensing device configured to monitor a pest trap; and
a computing device configured to determine, based on sensor information received from the at least one sensing device, a compliance status.

11. The system of claim 10, wherein sensor information comprises at least one of human behavioral data, environmental data, biological data, or machine data.

12. The system of claim 10, further comprising a data store configured to store data comprising the sensor information and rules information.

13. The system of claim 12, wherein the rules information comprises at least one of a governmental regulation, an industry standard, or a company specification.

14. The system of claim 12, wherein the computing device determines the compliance status by determining whether the sensor information complies with the rules information.

15. The system of claim 12, wherein the rules information comprises at least one instruction or task for a human worker.

16. The system of claim 10, wherein the at least one sensing device comprises a motion sensor that detects motion in or around the pest trap.

17. The system of claim 10, wherein:
the at least one sensing device comprises an imaging device that captures images in or around the pest trap; and
the computing device is further configured to perform image processing on the captured images, wherein the image processing comprises:
recognizing an image pattern corresponding to the pest trap; and
determining at least one property of the pest trap.

18. The system of claim 17, wherein the imaging device comprises an infrared sensor and the at least one property indicates a temperature in or around the pest trap.

19. The system of claim 17, wherein the imaging device comprises an optical imaging device, and the at least one property indicates a physical configuration of the pest trap.

20. The system of claim 19, wherein the optical imaging device is further configured to coordinate the timing of image capturing with at least one illumination device.

21. The system of claim 12, wherein the computing device is configured to determine a location of the pest trap by accessing the data store to determine one of a plurality of predetermined geographic regions.

22. The system of claim 10, further comprising a plurality of receivers, and wherein the computing device determines a location of the pest trap based, at least in part, on signals received from the plurality of receivers.

23. The system of claim 10, wherein the at least one sensing device is configured to be periodically polled for updates.

24. The system of claim 10, further comprising a display device configured to present a display indicating the status of pest infestation.

25. At least one computer-readable medium having stored thereon computer-readable program instructions which, when executed by at least one processor, perform acts of:
receiving sensor data indicating activation of a pest trap;
processing at least part of the sensor data;
determining, based on the processing of at least part of the sensor data, whether the sensor data satisfies at least one rule; and
outputting at least one result based on determining whether the sensor data satisfies the at least one rule.

26. The at least one computer-readable medium claim 25, wherein sensor data comprises at least one of human behavioral data, environmental data, biological data, or machine data.

27. The at least one computer-readable medium claim 25, wherein the at least one rule comprises at least one of a governmental regulation, an industry standard, or a company specification.

28. The at least one computer-readable medium claim 25, wherein the at least one rule comprises at least one instruction or task for a human worker.

29. The at least one computer-readable medium claim 25, wherein the sensor data indicates motion in or around the pest trap.

30. The at least one computer-readable medium claim 25, wherein:
the sensor data comprises images of regions in or around the pest trap; and
processing at least part of the sensor data comprises performing image processing of the captured images, wherein the image processing comprises:
recognizing an image pattern corresponding to the pest trap; and
determining at least one property of the pest trap.

31. The at least one computer-readable medium claim 30, wherein the images comprise infrared images and the at least one property indicates a temperature in or around the pest trap.

32. The at least one computer-readable medium claim 30, wherein the images comprise optical images, and the at least one property indicates a physical configuration of the pest trap.

33. The at least one computer-readable medium claim 32, wherein the optical images are further configured to be coordinated with timing of at least one illumination device.

34. The at least one computer-readable medium claim 25, wherein processing at least part of the sensor data comprises determining a location of the pest trap by accessing a data store to determine one of a plurality of predetermined geographic regions.

35. The at least one computer-readable medium claim 25, wherein processing at least part of the sensor data comprises determining a location of the pest trap based, at least in part, on signals received from a plurality of receivers.

36. A system configured to monitor, manage, and instrument compliance in a distributed work environment, the system comprising:
at least one input configured to receive data comprising pest infestation information and secondary information related to one or more of
a. behavior of one or more persons responsible for taking action in the distributed work environment,
b. biological or environmental parameters associated with the distributed work environment,
c. operational conditions and/or events,
d. apparatus usage and/or condition,
e. at least one standard and degree of compliance therewith, or
f. product production and/or delivery logistics;
a data store configured to store the data;
at least one processor configured to execute stored program instructions to process at least part of the data;
determine, based on the processing of at least part of the data, a compliance status.

37. The system of claim 36, wherein the compliance status comprises at least one of a pest trap status, a behavioral status, a biological status, an operational status, a machine status, or a logistical status.

38. The system of claim 36, wherein determining the compliance status comprises determining whether at least one entity has satisfied a requirement related to pest infestation.

39. The system of claim 36, wherein processing at least part of the data comprises determining at least one pest trap associated with the pest infestation information.

40. The system of claim 36, wherein the pest infestation information indicates either an individual pest trap, a group of pest traps, or a region of pest traps.

41. The system of claim 36, wherein the at least one input is further configured to receive the data using at least one sensor.

42. The system of claim 41, wherein the at least one sensor is configured to detect an activation status of at least one pest trap.

43. The system of claim 41, wherein the at least one sensor is configured to detect a human behavior comprising at least one of a speech, a motion, a location, or an interaction.

44. The system of claim 41, wherein the at least one sensor is dynamically re-configurable based, at least in part, on processing at least part of the data.

45. The system of claim 36, wherein the at least one standard comprises at least one of a governmental regulation, an industry standard, or a company specification.

46. The system of claim 36, wherein determining the compliance status comprises estimating a current and/or future state of the compliance status, based on the processing of the at least part of the data.

47. The system of claim 36, wherein determining the compliance status comprises determining whether at least one requirement has been satisfied by an entity involved in the distributed work environment.

48. The system of claim 47, wherein determining whether at least one requirement has been satisfied by an entity comprises comparing the data with a predetermined pattern, to determine an indication of a fraudulent and/or erroneous activity.

49. The system of claim 36, further comprising an output device configured to present an output result indicating the compliance status.

50. The system of claim 49, wherein the output result further comprises at least one instruction.
